(19) United States
(12) Patent Application Publication
(10) Pub. No.: US 2010/0323062 A1
(43) Pub. Date: Dec. 23, 2010

(54) BIOPROCESSING OF GRAINS

(75) Inventors: Michael David Southan, Manly Vale (AU); John Ronald Bradner, Pymble (AU); Robert Drant Willows, Marrickville (AU); Thomas Hugh Roberts, Putney (AU); Brian James Atwell, Lane Cove (AU)

Correspondence Address: LEYDIG VOIT & MAYER, LTD, TWO PRUDENTIAL PLAZA, SUITE 4900, 180 NORTH STETSON AVENUE, CHICAGO, IL, 60601-6731, US

(73) Assignee: GRAIN FOODS CRC LTD, North Ryde, New South Wales (AU)

(21) Appl. No.: 12/293,685

(22) PCT Filed: Mar. 21, 2007
(86) PCT No.: PCT/AU07/00348

§ 371 Date: Sep. 19, 2008

(30) Foreign Application Priority Data
Mar. 21, 2006 (AU) 2006901437

(51) Int. Cl. A23L 1/105; A21D 2/00; C12N 9/00; C12N 9/20; C12N 9/42; C07K 14/415

(52) U.S. Cl. 426/28; 426/622; 435/183; 435/198; 435/209; 530/370

(57) ABSTRACT

A method of treating a crop kernel prior to milling to improve millability, which includes the step of exposing the crop kernel to one or more plant hormones is provided. Typically, the crap kernel is a cereal such as wheat. The plant hormone is selected from the group consisting of auxins, gibberellins and abscisic acid. The method further includes the step of exposing the crop kernel to an enzyme. Typically the enzyme is a plant cell-wall degrading enzyme such as xylanase, lipase and cellulase. Also provided are methods of production of flour, food products and compositions. A particular application of this method is the optimisation of milling performance for the production of high quality flour.

### FIELD OF THE INVENTION

THIS INVENTION relates to milling of crop kernels. More particularly, this invention relates to an improved process of milling grains which produces high quality flour in high yields.

### BACKGROUND OF THE INVENTION

Milling of crop kernels for the production of flour has evolved from a primitive process of grinding kernels between two stones to a highly mechanised and commercially important process. However, the primary objective of milling has remained constant: separation of the kernel into its basic constituents and the grinding of one or more of those constituents into a fine powder. This process involves a number of steps. Initially, the crop kernel is “cleaned” in order to remove large foreign matter such as dirt, stones, leaves etc prior to conditioning of the kernel. Following conditioning, the kernel is passed through several rounds of breakage, sifting, purification and reduction until a fine powder is produced.

The practice of conditioning (or tempering) the crop kernel, an essential part of the milling process, typically involves adding a certain amount of moisture to the kernel then allowing it to lie for a time so that optimum milling performance will be obtained (i.e. achievement of maximum yield of flour with minimal bran contamination). In the case of wheat, the level of moisture added to the grain depends on whether the wheat is hard or soft, with hard wheats generally conditioned to 15.5 to 17% moisture content and soft wheats to 14 to 15.5% moisture content. The lying time at ambient temperatures between damping and milling usually ranges from 8 to 18 hours although commercial pressures may result in lying times occurring outside this range.

There are two basic objectives for conditioning wheat: the endosperm should be friable and readily reduced while the bran should remain tough and resistant to fragmentation. At high moisture levels the endosperm loses its friability while at low moisture levels bran becomes brittle and is readily abraded. In practice, conditioning represents a compromise between these extremes.

Therefore, wheat conditioning is essential for optimal milling performance and separation of the outer bran layers from the inner endosperm during the grinding process thereby maximising the yield of flour whilst minimising bran contamination. However because of the mechanical shear forces associated with the milling process some bran contamination in the flour is inevitable, particularly in high extraction or ‘straight run’ flours. For the milling industry to produce high quality flours with very low bran contamination significant flour yield is sacrificed i.e. flour yields are reduced from 78% to 60% or even as low as 40%.

The flour milling industry avoids using germinated or sprouted wheat because of the deleterious effects on flour quality. This is the reason why grain growers receive a lower payment for wheat that has been weather damaged. The degree of damage is moderated by the time over which conditions are wet. It is the duration the grain is moist that controls the extent of biochemical change.

The conditioning process (and the early stages of malting in barley) simulates a light rainfall on mature wheat. This is evidenced by the decrease in test weight of wheat after conditioning; the bran layers swell but they do not shrink back to their original size.

Germination requires enzyme-catalysed metabolic changes, many of which are regulated by endogenous plant hormones. Some of these biological processes are tissue-specific; some enzymes break down storage compounds while others synthesise new tissues.

International publication WO 02/00910 refers to a process of treating crop kernels, in particular corn, for 1-48 hours in the presence of cell degrading enzymes including acidic proteases, xylanases, cellulases, arabinofuranosidases and lipolytic enzymes.

International publication WO 02/00731 refers to an improved process of wet milling of crop kernels which includes the step of treating the ground kernels with an acidic protease.

International publication WO 99/21656 refers to an improved conditioning process for grain by addition of an enzyme preparation.

### SUMMARY OF THE INVENTION

There exists a commercial need to optimise milling performance for the production of high quality flour without a decrease in quality and yield. The present inventors have developed an effective process for the production of high quality flour whilst minimising bran contamination without sacrificing high yields. A preferred advantage provided by the invention is a decrease in kernel preparation time.

In one broad form, the invention relates to use of one or more plant hormones in the production of flour.

In a first aspect, the invention provides a method of treating a crop kernel prior to milling, which includes the step of exposing the crop kernel to one or more plant hormones.

In a second aspect, the invention provides a method of producing flour which includes the step of treating a crop kernel with one or more plant hormones prior to milling.

A preferred object of the invention is a method of treating a crop kernel prior to milling to improve crop kernel millability wherein said method includes the step of exposing the crop kernel to one or more plant hormones, which thereby improves millability of the crop kernel.

In a preferred embodiment of the methods of the first and second aspects, the method further includes a step of treating the crop kernel with an enzyme.

Preferably, the enzyme is a plant cell wall-degrading enzyme.

More preferably, the plant cell wall-degrading enzyme, is selected from the group consisting of a xylanase, a cellulase and a lipase.

Even more preferably, the cell wall-degrading enzyme is a cellulase.

In a third aspect, the invention provides a flour produced according to the method of the second aspect.

In a fourth aspect, the invention provides a food product produced using the flour of the third aspect.

In a fifth aspect, the invention provides a composition for treating a crop kernel prior to milling comprising one or more plant hormones of the first aspect with a suitable carrier or diluent.

Preferably, the crop kernel comprises at least an endosperm and a bran layer.

In a particular embodiment, the crop kernel is a grain such as wheat.

Preferably, the crop kernel is treated for a period between 1-24 hours.

More preferably, the crop kernel is treated for a period between 8 and 18 hours.

Even more preferably, the crop kernel is treated for a period between about 14-16 hours.

Preferably, the plant hormone is selected from the group consisting of a gibberellin, an abscisic acid and an auxin.

More preferably, the plant hormone is abscisic acid.

Preferably, the plant hormone is added to a final concentration between 0.5 and 50 mg/kg crop kernel.

More preferably, the plant hormone is added to a final concentration of between 1 and 20 mg/kg crop kernel.

Even more preferably, the plant hormone is added to a final concentration of about 2 mg/kg crop kernel.

In a particular preferred embodiment, the method includes the combined steps of exposing the crop kernel to a solution containing a plant hormone and a plant cell wall-degrading enzyme.

Throughout this specification, unless the context requires otherwise, the words “comprise”, “comprises” and “comprising” will be understood to imply the inclusion of a stated integer or group of integers but not the exclusion of any other integer or group of integers.

### BRIEF DESCRIPTION OF THE FIGURES

FIG. 1 Effect of plant hormones on flour yield. The data points are as follows: Circle is control, diamond is abscisic acid, square is gibberellic acid, triangle is indole acetic acid.

FIG. 2 Impact of the addition of cell wall-degrading enzymes on bran layers and endosperm. A=Control (water), B=Xylanase (100 mg/ml of diluent), C=Cellulase (100 mg/ml of diluent), D=Lipase (2 mg/ml of diluent).

FIG. 3 Effect of xylanase and cellulase on flour yield. The data points are as follows: circle is control; square is xylanase; triangle is cellulase.

FIG. 4 Effect of lipase on flour yield. Circle is control; square is lipase.

FIG. 5 Effect of xylanase and cellulase on dough strength. The light toned cross-hatched filled bars are control; the medium toned cross-hatched bars are xylanase; the dark toned cross-hatched bars are cellulase.

FIG. 6 Effect of lipase on flour paste viscosity. The light toned cross-hatched bars are control; the medium toned cross-hatched filled bars are lipase.

FIG. 7 Effect of conditioning additives on Rapid Dough Total Score. Treatment 1=abscisic acid (ABA) at 1.5 mg/kg crop kernel; Treatment 2=cellulase at 250 mg/kg crop kernel; Treatment 3=lipase at 100 mg/kg crop kernel. The solid filled bar is control; the diagonal filled bars are treatments.

FIG. 8 Effect of cellulase and abscissic acid on wheat flour yield.

FIG. 9 Effect of ABA at different concentrations and different wheat quantities on flour yield. 1 ppm=1 mg ABA per kg crop kernel.

### DETAILED DESCRIPTION OF THE INVENTION

The present inventors have developed an improved method to process crop kernels for the commercial production of flour. The product of this invention is enhanced flour yield with minimal bran contamination. The method of this invention selectively improves toughening of the outer bran layer of the grain, which aids in separation of the bran from endosperm, whilst softening the endosperm to assist with milling. The present invention overcomes a major disadvantage of conventional, prior art approaches to this important step in the milling process.

By “crop kernel” is meant a product of a crop such as a seed or a grain (although without limitation thereto) comprising an endosperm and a bran layer.

Flour can be milled from a variety of crops, primarily cereals or other starchy food sources. Non-limiting examples are wheat, corn, rye, rice, barley, as well as other grasses and seed producing crops such as legumes and nuts.

Preferably, the crop is a cereal.

Even more preferably, the cereal is wheat.

Different types of flour have varying proportions of grain constituents. For example, white flour is made from endosperm only whereas wholegrain flour is made from the entire grain and germ flour is made from the endosperm and germ. It follows that for the production of high quality white flour, a crucial step is to separate the bran layers and germ from the endosperm as efficiently as possible. The preferred method is to induce structural changes in the outer layers of the grain that are analogous to those that occur at the onset of germination. Preferably, germination is induced by exposing the grain to moisture.

Preferably, “exposing” the crop kernel can include steeping, soaking, immersing, saturating, wetting and spraying. More preferably, the crop kernel is wetted. In a preferred embodiment, the crop kernel is wetted such that the moisture content is between 14-17%.

The duration that the grain is exposed to moisture is an important variable as this controls the extent of biochemical change within the grain. If the grain is wet for a prolonged period of time, germination will proceed to completion, which renders the grain useless for milling. Preferably, the grain is exposed to moisture for between 1-24 hours. More preferably, the grain is exposed to moisture for between 8 and 18 hours. Even more preferably, the grain is exposed to moisture for between about 14 to about 16 hours.

Although not wishing to be bound by any particular theory, it is proposed that the onset of germination of grain can also be promoted by a variety of physical and/or chemical stimuli. Preferably, germination is promoted by a chemical stimulus. More preferably, germination is promoted by hormones. Even more preferably, germination is promoted by plant hormones.

By “plant hormones”, such as in the context of hormones utilised in this invention, it is meant any class of small organic molecule that regulates enzymatic activity or which alters the pattern of gene expression in plants. There are five major classes of plant hormones: auxins, cytokinins, gibberellins, abscisic acid and ethylene. It can be appreciated that a plant hormone may be derived from a variety of sources including a natural or chemical source. It can be contemplated that a synthetic analogue of a plant hormone may be used in the present invention.

Preferably, the plant hormone is selected from the group consisting of gibberellins, abscisic acid and auxins.

More preferably, the plant hormone is abscisic acid added to a final concentration between 0.5 and 50 mg/kg crop kernel. Even more preferably, the plant hormone is added to a final concentration between 1 and 20 mg/kg crop kernel. In particular preferred embodiments, the plant hormone concentration is added to a final concentration of 1, 1.5, 2.0, 2.5, 3.0, 3.5, 4.0, 4.5, 5.0, 5.5, 6.0, 6.5, 7.0, 8.0, 9.0, 10, 12, 14, 16, 18 or 20 mg/kg crop kernel.

In a preferred embodiment, abscisic acid is added to a final concentration of about 2 mg/kg crop kernel.

Therefore one broad form of this invention is a method for treating a crop kernel prior to milling to improve crop kernel millability, where the method includes the step of exposing the crop kernel to one or more plant hormones, which thereby improves millability.

By “millability” is meant the capability of a crop kernel to be milled into a flour. The millability of a crop kernel is related to kernel hardness, the endosperm to bran ratio and ease of separation of the bran but is not limited thereto. Typically, although not exclusively, the milling process is more straightforward if the starting material exhibits a readier separation of bran from endosperm as the resultant flour is more mobile and easier to sift. Generally, optimum millability is the achievement of maximum yield of flour with minimal bran contamination. Throughout this specification, millability will be used interchangeably with “milling performance”.

It will be appreciated by a person of skill in the art that the method of the present invention can be applied to a conventional flour mill apparatus.

In a preferred embodiment of the invention, an enzyme may be added to the process. The purpose of adding an enzyme is to assist release of the endosperm during milling. Most suitably, the enzyme is a plant cell wall-degrading enzyme. Non-limiting examples of such enzymes include pentosanases, fructanases, arabinases, mannosidases, cellulases, xylanases and lipolytic enzymes. Preferably, the enzymatic activity is chosen from the group consisting of xylanases, cellulases and lipolytic enzymes. More preferably, the enzyme is a cellulase.

Preferably, the enzyme is added to final concentration of between 50 and 1000 mg/kg crop kernel. More preferably, the enzyme is added to a final concentration of between 100 and 500 mg/kg crop kernel. In particular preferred embodiments, the enzyme is added to final concentration of 100, 110, 120, 130, 140, 150, 160, 170, 180, 190, 200, 210, 220, 230, 240, 250, 260, 270, 280, 290, 300, 310, 320, 330, 340 or 350 mg/kg crop kernel.

In a preferred embodiment, the enzyme is added to a final concentration of about 250 mg/kg crop kernel.

Typically, plant cell wall-degrading enzymes are derived from either fungal or bacterial organisms however it may be contemplated that the enzyme is derived by recombinant methodology.

A recombinant enzyme may be conveniently prepared by a person skilled in the art using standard protocols as for example described in Sambrook and Russell, MOLECULAR CLONING. A Laboratory Manual (3^{rd} edition) (Cold Spring Harbor Laboratory Press, New York), incorporated herein by reference, in particular Sections 16 and 17; CURRENT PROTOCOLS IN MOLECULAR BIOLOGY Eds. Ausubel et al., (John Wiley & Sons, Inc. 1995-1999), incorporated herein by reference, in particular Chapters 10 and 16; and CURRENT PROTOCOLS IN PROTEIN SCIENCE Eds. Coligan et al., (John Wiley & Sons, Inc. 1995-1999) which is incorporated by reference herein, in particular Chapters 1, 5 and 6.

It will be appreciated by the foregoing that in a preferred embodiment, the invention provides a method of treating a wheat kernel prior to milling to improve wheat kernel millability, said method including the step of exposing the wheat kernel for a period between about 14 and about 16 hours with an abscisic acid at a final concentration of about 2 mg/kg crop kernel and a cellulase at a final concentration of about 250 mg/kg crop kernel, which thereby improves the millability of said wheat kernel.

It is preferable to administer the composition to the grain by means of a solution. More preferably, the crop kernel is exposed to an aqueous solution containing the plant hormone and plant cell wall-degrading enzyme.

In a preferred embodiment, the invention provides a composition for treating a wheat kernel prior to milling to improve millability, wherein said composition is a solution comprising an abscisic acid at a final concentration of about 2 mg/kg crop kernel, a cellulase at a final concentration of about 250 mg/kg crop kernel and a suitable carrier or diluent.

It can readily appreciated that flour produced using the present invention has application in the manufacture of baked goods such as bread, pastries, biscuits, cakes and other food stuffs such as Asian noodles, Chinese steamed breads, Middle Eastern flat breads, pasta and some confectionary such as liquorice. A further use for flour includes as a yeast food for brewing beer.

Two of the most important constituents of flour, starch and gluten, have a variety of applications in the food industry and beyond. For example, starch is used as cornflour or may be converted into glucose and other sugars for use in the production of confectionary and other foods. Starch also forms a basic ingredient of adhesives and gums. The binding and water absorption properties of gluten make it an important ingredient in smallgoods, bread and textured vegetable protein products.

So that the present invention may be more readily understood and put into practical effect, the skilled person is referred to the following non-limiting examples.

### EXAMPLES

### Example 1

### Laboratory Scale Milling Incorporating Plant Hormones

### Materials and Methods

Wheat cv. Wedgetail was milled on a laboratory Buhler test mill to determine whether the addition of any of the major plant hormones i.e. gibberellic acid (GA₃), indole acetic acid (IAA), or absicisic acid (ABA) had an impact on flour yield or flour quality.

A matrix design experiment was conducted where all the three hormones at 1.5 mg/kg crop kernel concentration plus control samples were milled at nominal times after conditioning of 12, 16, 20 and 24 hours. The standard conditioning time for hard wheat such as Wedgetail is 16 hours.

### Results and Discussion

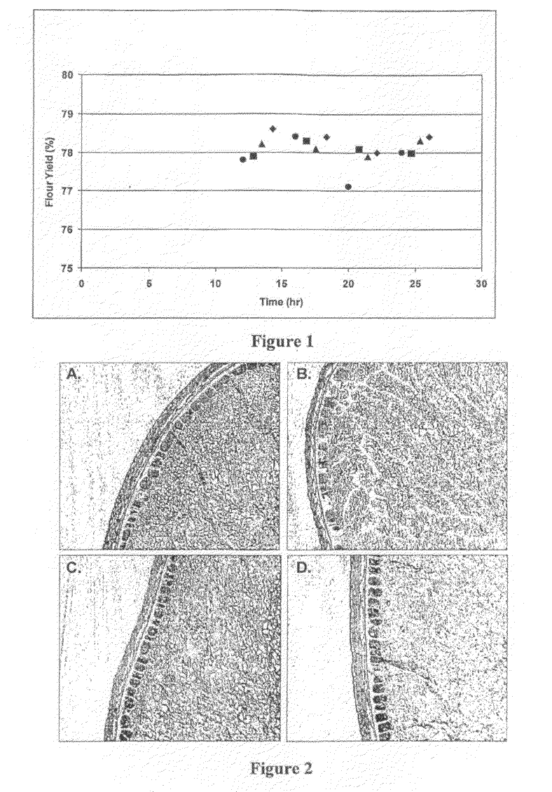

The results of this test are shown in Table I and in a graphic form in FIG. 1. Flour yield was highest for the control samples after a 16 hour conditioning time. Interestingly, the highest flour yield resulted from the ABA treatment and after only a 14 hour conditioning time. ABA produced the highest or equal to highest flour yields for the 12, 16, 20 and 24 hour conditioning intervals.

All samples were milled on the same mill and by the same operator and treatments were milled in the same order for each time point to minimise differences due to mill temperature changes. Flour analysis including flour moisture, bran, protein and ash content, starch damage, colour grade, flour Minolta colour, water absorption, dough development time, stability, extensibility, dough strength and flour viscosity were not adversely affected by the hormone treatments.

### Conclusions

The above results demonstrate that treatment of wheat during conditioning with 1.5 mg ABA per kg crop kernel appears to increase flour yield slightly and reduce conditioning times. The potential commercial value is to increase flour yields without adversely affecting flour quality and with shorter conditioning times.

### Example 2

### Impact of Enzymes on Cellular Structure, Flour Yield and Quality

### Materials and Methods

The effect of enzymes on cellular structure was investigated by standard light microscopy techniques. The grain kernels were sectioned on a microtome, stained and viewed under a light microscope.

Wheat cv. Wedgetail was milled on a laboratory Buhler test mill to determine whether the enzymes identified as having an effect on the grain structure by microscopy had an impact on flour yield or flour quality.

A matrix design experiment was conducted where cellulase and xylanase at 250 mg/kg crop kernel respectively and lipase at 100 mg/kg crop kernel plus control samples were milled at nominal times of 12, 16, 20 and 24 hours after conditioning. The standard conditioning time for hard wheat such as Wedgetail is 16 hours.

### Impact of Cell Wall Degrading Enzymes on Cellular Structure

In FIG. 2, A to D the impact of the addition of xylanase, cellulase and lipase on both the bran layers and the endosperm is observed. Furthermore, when the enzyme concentrations were reduced five-fold (compared to the concentrations used in FIG. 2), the effect is still apparent. Of particular interest is the effect on the bran layers and aleurone cells generated by the addition of the commercial lipase preparation. Under higher magnification there is a strong indication that the bran layers are more ‘relaxed’ than those seen in the control. Additionally, the disruption of the aleurone cells, suggest the presence of a mechanical weakness in these cells not apparent under normal conditions.

### The Impact of Addition of Cell Wall Degrading Enzymes on Flour Yield

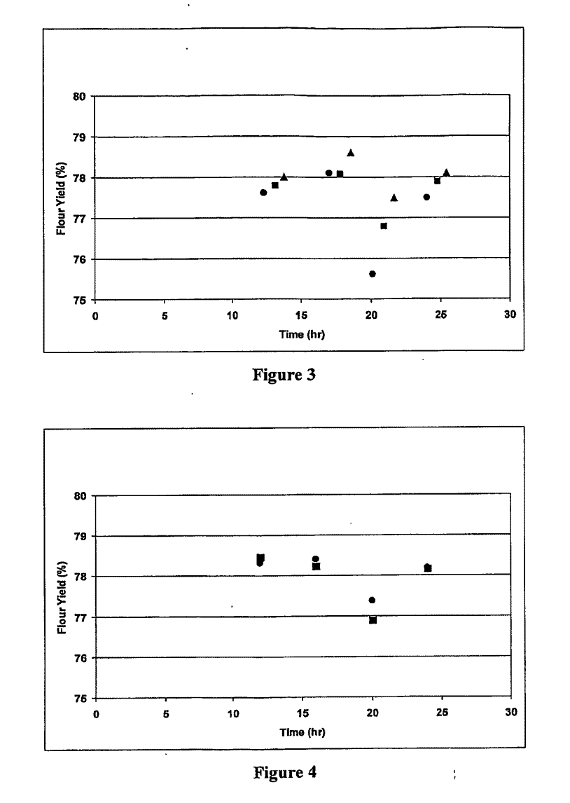

The impact of each enzyme during conditioning on flour yield is shown in FIGS. 3 and 4. Cellulase provided the greatest increase in flour yield for the enzyme treatments between 12 and 24 hours after conditioning. As cellulase had the greatest impact on flour yield, two sources of cellulase were compared: one food grade cellulase from Westons and one non-food grade cellulase from Macquarie University. The two enzyme samples added in the concentrations which produced similar activities produced similar increases in flour yield over the control after 16 hours conditioning.

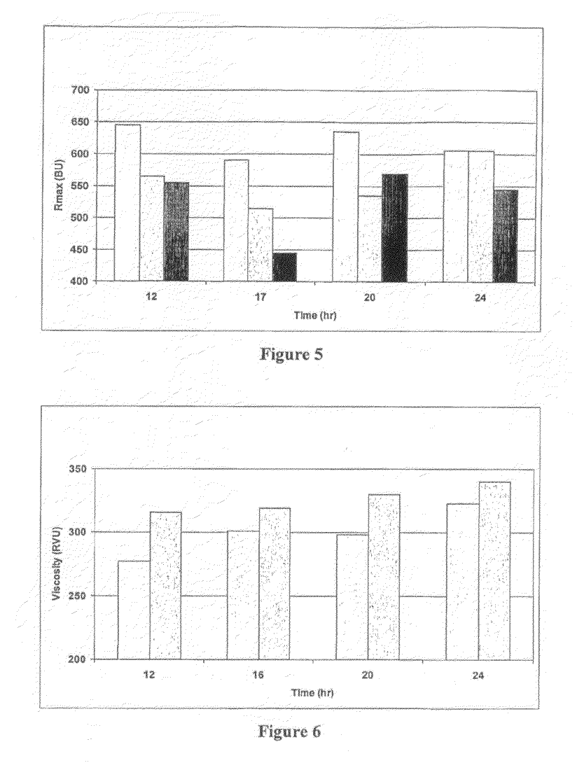

The flour quality of each of the enzyme treatments was tested. It can be clearly seen that cellulase treatment decreased the dough strength of a strong flour (FIG. 5) whereas lipase treatment increased flour viscosity (FIG. 6).

### Example 3

### Impact of Enzymes on End Product Quality

Flours from cv. Wedgetail that was milled on a laboratory Buhler test mill with either cellulase added at 250 mg/kg crop kernel, lipase added at 100 mg/kg crop kernel or ABA at 1.5 mg/kg crop kernel plus control samples were test baked as rapid doughs to determine the impact of enzyme treatment on baking quality.

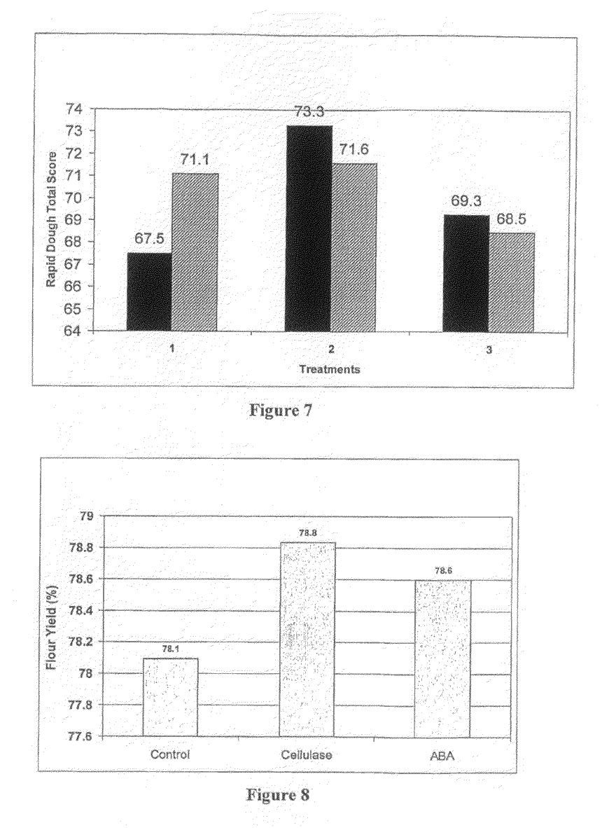

The impact of each enzyme and ABA during conditioning on baking quality is shown in FIG. 7. The range of scores for the controls was 67.5 to 73.3. The rapid dough scores after the treatments were added to the conditioning water was within this range i.e. 68.5 to 71.6. The average control score was 70.0. The ABA and cellulase treatments scored slightly higher than the average control score. This indicates that the treatments which increased flour yield ie. ABA and cellulase do not adversely affect baking quality.

### Example 4

### Increase in Flour Yield Over Many Observations Using ABA

The data represented in FIG. 8 builds on the data presented above in that values in this graph represented by the bars are averages of 9 observations for the control samples; 5 observations for the cellulase treated samples and 4 observations for the ABA treated samples.

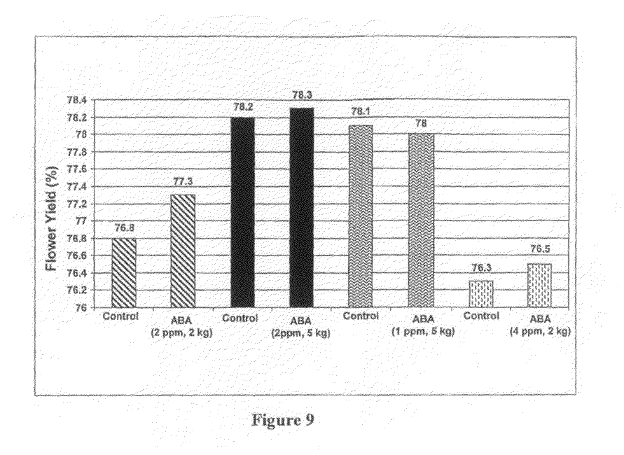

FIG. 9 shows the increase in flour yield when 2 mg ABA per kg crop kernel is used on wheat over several observations. The diagonal filled bars are average values for 6 observations; the solid black bars are average values for 4 observations; the wave filled bars are average values for 4 observations; the vertical dashed filled bars are average values for 2 observations. Moreover at 2 mg/kg crop kernel, flour yield increases when the wheat sample milled is 2 kg or 5 kg even though the control sample flour yields are higher for the 5 kg samples. No increase in flour yield was observed for 1 mg/kg crop kernel. At 4 mg/kg crop kernel flour yield increased.

Throughout this specification, the aim has been to describe the preferred embodiments of the invention without limiting the invention to any one embodiment or specific collection of features. Various changes and modifications may be made to the embodiments described and illustrated herein without departing from the broad spirit and scope of the invention.

All computer programs, algorithms, scientific and patent literature described in this specification are incorporated herein by reference in their entirety.

### Tables

TABLE I
The effect of plant hormones at various conditioning
times on flour yield and recovery

| Treatment | Conditioning Time (hr) | Flour Yield (%) | Recovery (%) |
|---|---|---|---|
| Control | 12.08 | 77.8 | 99.6 |
| Control | 16.05 | 78.4 | 96.8 |
| Control | 20.00 | 77.1 | 96.3 |
| Control | 24.00 | 78.0 | 97.1 |
| GA3 | 12.83 | 77.9 | 97.5 |
| GA3 | 16.83 | 78.3 | 95.9 |
| GA3 | 20.75 | 78.1 | 98.8 |
| GA3 | 24.67 | 78.0 | 95.8 |
| IAA | 13.50 | 78.2 | 97.0 |
| IAA | 17.58 | 78.1 | 96.3 |
| IAA | 21.45 | 77.9 | 96.2 |
| IAA | 25.33 | 78.3 | 96.8 |
| ABA | 14.33 | 78.6 | 96.6 |
| ABA | 18.37 | 78.4 | 95.7 |
| ABA | 22.12 | 78.0 | 96.4 |
| ABA | 26.03 | 78.4 | 96.3 |

1. A method of treating a crop kernel prior to milling to improve crop kernel millability, said method including the step of exposing the crop kernel to one or more plant hormones which improves millability of the crop kernel.

2. The method of claim 1, wherein the crop kernel comprises at least an endosperm and a bran layer.

3. The method of claim 2, wherein the crop kernel is a cereal.

4. The method of claim 3, wherein the cereal is wheat.

5. The method of claim 1, wherein the crop kernel is treated for a period between 1 and 24 hours.

6. The method of claim 5, wherein the crop kernel is treated for a period between 8 and 18 hours.

7. The method of claim 6, wherein the crop kernel is treated for a period between about 14 and about 16 hours.

8. The method of claim 1, wherein the crop kernel has a moisture content of between 14 and 17%.

9. The method of claim 1, wherein the one or more plant hormones are selected from the group consisting of an auxin, a gibberellin and an abscisic acid.

10. The method of claim 9, wherein the plant hormone is abscisic acid only.

11. The method of claim 1, wherein the plant hormone is added to a final concentration of between 0.5 and 50 mg/kg crop kernel.

12. The method of claim 11, wherein the final concentration of the plant hormone is between 1 and 20 mg/kg crop kernel.

13. The method of claim 12, wherein the final concentration of the plant hormone is about 2 mg/kg crop kernel.

14. The method of claim 1, said method further including the step of exposing the crop kernel to an enzyme.

15. The method of claim 14, wherein the enzyme is a plant cell wall-degrading enzyme.

16. The method of claim 15, wherein the plant cell wall-degrading enzyme is selected from the group consisting of a xylanase, a cellulase and a lipase.

17. The method of claim 16, wherein the plant cell wall-degrading enzyme is a cellulase.

18. The method of claim 14, wherein a final concentration of the enzyme is between 50 and 1000 mg/kg crop kernel.

19. The method of claim 18, wherein the final concentration of the enzyme is between 100 and 500 mg/kg crop kernel.

20. The method of claim 19, wherein the final concentration of the enzyme is about 250 mg/kg crop kernel.

21. A method of treating a wheat kernel prior to milling to improve wheat kernel millability, said method including the step of exposing the wheat kernel for a period between about 14 and about 16 hours with an abscisic acid at a final concentration of about 2 mg/kg crop kernel and a cellulase at a final concentration of about 250 mg/kg crop kernel, which thereby improves the millability of said wheat kernel.

22. A method of producing a flour, wherein said method includes the step of treating a crop kernel prior to milling according to claim 1.

23. A flour produced according to the method of claim 22.

24. A food product produced using the flour of claim 23.

25. A composition when used for treating a crop kernel prior to milling to improve millability, said composition comprising one or more plant hormones and a suitable carrier or diluent.

26. The composition of claim 25, wherein the one or more plant hormones are selected from the group consisting of an auxin, a gibberellin and an abscisic acid.

27. The composition of claim 26, wherein the plant hormone is an abscisic acid only.

28. The composition of claim 26, wherein the plant hormone is added to a final concentration of between 0.5 and 50 mg/kg crop kernel.

29. The composition of claim 28, wherein the final concentration is between 1 and 20 mg/kg crop kernel.

30. The composition of claim 29, wherein the final concentration is about 2 mg/kg crop kernel.

31. The composition of claim 25, wherein the composition further comprises an enzyme.

32. The composition of claim 31, wherein the enzyme is a plant cell wall-degrading enzyme.

33. The composition of claim 32, wherein the plant cell wall-degrading enzyme is selected from the group consisting of a xylanase, a lipase and a cellulase.

34. The composition of claim 33, wherein the plant cell-wall degrading enzyme is a cellulase.

35. The composition of claim 31, wherein a final concentration of an enzyme is between 50 and 1000 mg/kg crop kernel.

36. The composition of claim 35, wherein the final concentration of an enzyme is between 100 and 500 mg/kg crop kernel.

37. The composition of claim 36, wherein the final concentration of an enzyme is about 250 mg/kg crop kernel.

38. The composition of claim 26, which is a solution.

39. A composition for treating a wheat kernel prior to milling to improve millability, wherein said composition is a solution comprising an abscisic acid at a final concentration of about 2 mg/kg crop kernel, a cellulase at a final concentration of about 250 mg/kg crop kernel and a suitable carrier or diluent.
